(19) United States
(12) Patent Application Publication
(10) Pub. No.: US 2015/0231134 A1
(43) Pub. Date: Aug. 20, 2015

(54) Combination Therapy using Belinostat and Trabectedin

(71) Applicant: Onxeo DK, Branch of Onxeo S.A., France, Copenhagen (DK)

(72) Inventor: Kamille Dumong Erichsen, Copenhagen (DK)

(21) Appl. No.: 14/428,188

(22) PCT Filed: Sep. 26, 2013
(86) PCT No.: PCT/IB2013/058891

§ 371 Date: Mar. 13, 2015

Related U.S. Application Data
(60) Provisional application No. 61/707,063, filed on Sep. 28, 2012.

(30) Foreign Application Priority Data
Sep. 28, 2012 (GB) 1217439.7

(51) Int. Cl. A61K 31/4995; A61K 31/18

(52) U.S. Cl. CPC A61K 31/4995; A61K 31/18

(57) ABSTRACT

The present invention relates generally to therapies for the treatment of diseases and disorders that are mediated by histone deacetylase (HDAC) (for example, cancer, including, for example, ovarian cancer, soft-tissue sarcoma, osteosarcoma, etc.) which employ a combination (e.g., a synergistic combination) of (a) belinostat, or a salt, hydrate, or solvate thereof, and (b) trabectedin, or a salt, hydrate, or solvate thereof.

### RELATED APPLICATIONS

This application is related to United Kingdom patent application number 1217439.7 filed 28 Sep. 2012 and U.S. provisional patent application No. 61/707,063 filed 28 Sep. 2012, the contents of both of which are incorporated herein by reference in their entirety.

### TECHNICAL FIELD

The present invention relates generally to therapies for the treatment of diseases and disorders that are mediated by histone deacetylase (HDAC) (for example, cancer, including, for example, ovarian cancer, soft-tissue sarcoma, osteosarcoma, etc.) which employ a combination (e.g., a synergistic combination) of (a) belinostat, or a salt, hydrate, or solvate thereof, and (b) trabectedin, or a salt, hydrate, or solvate thereof.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

Soft-Tissue Sarcoma

A soft-tissue sarcoma (STS) is a form of sarcoma that develops in connective tissue. They are relatively uncommon cancers and account for less than 1% of all new cancer cases each year. This may be because cells in soft-tissue, in contrast to tissues that more commonly give rise to malignancies, are not continuously dividing cells. In general, treatment for soft-tissue sarcomas depends on the stage of the cancer. The stage of the sarcoma is based on the size and grade of the tumour, and whether the cancer has spread to the lymph nodes or other parts of the body (metastasized). Treatment options for soft-tissue sarcomas include surgery, radiation therapy, and chemotherapy. Soft-tissue sarcoma can be divided into types depending upon the site of occurrence and histological features—the major subdivisions are: fibrosarcoma, myxofibrosarcoma, desmoid tumour, liposarcoma, synovial sarcoma, rhabdomyosarcoma, leiomyosarcoma, malignant peripheral nerve sheath tumours, angiosarcoma, gastrointestinal stromal tumour, Kaposi's sarcoma, Ewing's tumour, lyeolar soft part sarcoma, dermatofibromasarcoma protuberans, desmoplastic small round cell tumours, epithelioid sarcoma, extraskeletal myxoid chondrosarcoma, and giant cell fibrosarcoma.

Although small tumours can be successfully removed by surgery, patients with advanced soft-tissue sarcoma have a very poor prognosis, even with currently available chemotherapeutic regimens. Consequently, there remains a large unmet medical need for new drug treatments for Soft-tissue sarcoma including optimised novel combinations of known drugs.

Osteosarcoma

Osteosarcoma is an aggressive malignant neoplasm arising from primitive transformed cells of mesenchymal origin that exhibit osteoblastic differentiation and produce malignant osteoid. It is the most common histological form of primary bone cancer. Osteosarcoma is the eighth most common form of childhood cancer, comprising 2.4% of all malignancies in paediatric patients. Deaths due to malignant neoplasms of the bones and joints account for an unknown amount of childhood cancer deaths. Mortality rates due to osteosarcoma have been declining at approximately 1.3% per year; long-term survival probabilities for osteosarcoma have improved dramatically during the late 20th century and was approximately 68% in 2009. Despite the success of chemotherapy for osteosarcoma, it has one of the lowest survival rates for paediatric cancer. The best reported 10-year survival rate is 92%; the protocol used is an aggressive intra-arterial regimen that individualizes therapy based on arteriographic response. Three-year event-free survival ranges from 50% to 75%, and five-year survival ranges from 60% to 85+% in some studies. Overall, 65-70% patients treated five years ago will be alive today. Nonetheless, there remains a large unmet medical need for new drug treatments for osteosarcoma including optimised novel combinations of known drugs.

Ovarian Cancer

Ovarian cancer is the fourth leading cause of cancer deaths among women in the United States and causes more deaths than all the other gynaecologic malignancies combined.

In the United States, a woman's lifetime risk of developing ovarian cancer is 1 in 70. In 1992, about 21,000 cases of ovarian cancer were reported, and about 13,000 women died from the disease. See, e.g., Chapter 321, Ovarian Cancer, Harrison's Principles of Internal Medicine, 13^{th} ed., Isselbacher et al., eds., McGraw-Hill New York (1994), pages 1853-1858; and American Cancer Society Statistics, Cancer J. Clinicians, Vol. 45, No. 30 (1995).

Epithelial ovarian cancer, the most common ovarian cancer, has a distinctive pattern of spread in which cancer cells migrate throughout the peritoneal cavity to produce multiple metastatic nodules in the visceral and parietal peritoneum and the hemi diaphragms. In addition, metastasis can occur to distant sites such as the liver, lung and brain. Early stage ovarian cancer is often asymptomatic and is detected coincidentally by palpating an ovarian mass on pelvic examination. In premenopausal patients, about 95% of these masses are benign. Even after menopause, 70% of masses are benign but detection of any enlargement requires evaluation to rule out malignancy. In postmenopausal women with pelvic mass, a markedly elevated serum CA-125 level of greater than 65 U/mL indicates malignancy with 96% positive predictive value. See, e.g., Chapter 321, Ovarian Cancer, Harrison's Principles of Internal Medicine, 13^{th} ed., Isselbacher et al., eds., McGraw-Hill New York (1994).

Epithelial ovarian cancer is seldom encountered in women less than 35 years of age. Its incidence increases sharply with advancing age and peaks at ages 75 to 80, with the median age being 60 years. The single most important risk factor for this cancer is strong family history of breast or ovarian cancer. Oncogenes associated with ovarian cancers include the HER-2/neu (c-erbB-2) oncogene, which is over expressed in a third of ovarian cancers, the fms oncogene, and abnormalities in the p53 gene, which are seen in about half of ovarian cancers. A number of environmental factors have also been associated with a higher risk of epithelial ovarian cancer, including a high fat diet and intake of lactose in subjects with relatively low tissue levels of galactose-1-phosphate uridyl transferase.

The internationally-accepted first-line chemotherapy for advanced epithelial ovarian cancer is the combination of carboplatin and paclitaxel. Typical results are median progression-free survival (PFS) of 17-20 months and median survival of 3-5 years. Second-line treatment is determined by duration of remission. If relapse occurs within 6 months of the last treatment, patients are considered “platinum resistant”. Re-treatment with a carboplatin/paclitaxel regimen in these patients is associated with a low response rate (15%) of short duration (3-6 months), and a median survival of approximately 12 months.

Consequently, there remains a large unmet medical need for new drug treatments for ovarian cancer, especially epithelial ovarian cancer, including optimised novel combinations of known drugs.

Belinostat

Belinostat (CAS 414864-00-9) (also known as (E)-N-hydroxy-3-(3-phenylsulfamoyl-phenyl)-acrylamide, PXD101, and PX 105684), shown below, is a well known histone deacetylase (HDAC) inhibitor. It was first described in Watkins et al., 2002. It is being developed for treatment of a range of disorders mediated by HDAC, and is the subject of a number of Phase I and Phase II trials for various cancers.

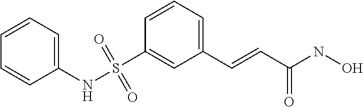 [SMILES: O=C(/C=C/c1cccc(S(=O)(=O)Nc2ccccc2)c1)NO]

Typically, liquid formulations of belinostat further comprise L-arginine, and are suitable for administration by injection, infusion, intravenous infusion, etc. See, for example, Bastin et al., 2006. Methods of treatment employing prolonged continuous infusion of belinostat are described, for example, in Sehested et al., 2009.

Trabectedin

Trabectedin (CAS 114899-77-3) (also known as ecteinascidin-743, ET-743, Yondelis®, and spiro[6,16-(epithiopropanoxymethano)-7,13-imino-12H-1,3-dioxolo[7,8]isoquino[3,2-b][3]benzazocine-20,1′(2′H)-isoquinolin]-19-one, 5-(acetyloxy)-3′,4′,6,6a,7,13,14,16-octahydro-6′,8,14-trihydroxy-7′,9-dimethoxy-4,10,23-trimethyl-, [6R-(6α,6aβ,7β,13β,14β,16α,20R*)]) is a marine alkaloid isolated from the Caribbean tunicate Ecteinascidia turbinate (see, e.g., Rinehart et al., 1987), with a chemical structure characterized by three fused tetrahydroisoquinoline rings.

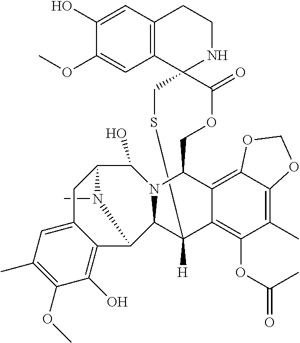

Two of these rings provide the framework for covalent interaction with the minor groove of the DNA double helix, whereas the third ring protrudes from the DNA duplex, apparently allowing interactions with adjacent nuclear proteins. The compound's chemical interactions trigger a cascade of events that interfere with several transcription factors, DNA binding proteins, and DNA repair pathways, likely to be different from other DNA-interacting agents. Trabectedin also causes modulation of the production of cytokines and chemokines by tumour and normal cells, suggesting that the antitumour activity could also be ascribed to changes in the tumour microenvironment. Trabectedin was approved in Europe in 2007 for soft-tissue sarcomas and in 2009 for ovarian cancer. It is currently marketed as Yondelis®. However, there is a great need for improvement of the prognosis of patients treated with trabectedin: for example, the time-to-tumour-progression (TTP) of patients with soft-tissue sarcoma treated with Yondelis® in the pivotal clinical trial was only 3.9 months.

### SUMMARY OF THE INVENTION

The present invention relates to the surprising and unexpected discovery that the combination of (a) belinostat, or a salt, hydrate, or solvate thereof and (b) trabectedin, or a salt, hydrate, or solvate thereof, is synergistic, for example, in the treatment of diseases and disorders which are mediated by histone deacetylase (HDAC) (for example, cancer, including, for example, ovarian cancer, soft-tissue sarcoma, osteosarcoma, etc.).

Thus, one aspect of the invention relates to a method of treatment, for example, of a disease or disorder which is mediated by histone deacetylase (HDAC) (for example, cancer, including, for example, ovarian cancer, soft-tissue sarcoma, osteosarcoma, etc.) in a patient in need of treatment, comprising administering to said patient (a) belinostat, or a salt, hydrate, or solvate thereof, and (b) trabectedin, or a salt, hydrate, or solvate thereof, in amounts such that the combination is therapeutically-effective (e.g., in amounts such that the combination is therapeutically synergistic).

Another aspect of the present invention relates to belinostat, or a salt, hydrate, or solvate thereof, for use, in combination with trabectedin, or a salt, hydrate, or solvate thereof, in a method of treatment of the human or animal body by therapy.

Another aspect of the present invention relates to trabectedin, or a salt, hydrate, or solvate thereof, for use, in combination with belinostat, or a salt, hydrate, or solvate thereof, in a method of treatment of the human or animal body by therapy.

Another aspect of the present invention relates to a combination of belinostat, or a salt, hydrate, or solvate thereof and trabectedin, or a salt, hydrate, or solvate thereof, for use in a method of treatment of the human or animal body by therapy.

Another aspect of the present invention relates to belinostat, or a salt, hydrate, or solvate thereof, for use, in combination with trabectedin, or a salt, hydrate, or solvate thereof, in a method of treatment, for example, of a disease or disorder which is mediated by histone deacetylase (HDAC) (for example, cancer, including, for example, ovarian cancer, soft-tissue sarcoma, osteosarcoma, etc.).

Another aspect of the present invention relates to trabectedin, or a salt, hydrate, or solvate thereof, for use, in combination with belinostat, or a salt, hydrate, or solvate thereof, in a method of treatment, for example, of a disease or disorder which is mediated by histone deacetylase (HDAC) (for example, cancer, including, for example, ovarian cancer, soft-tissue sarcoma, osteosarcoma, etc.).

Another aspect of the present invention relates to a combination of belinostat, or a salt, hydrate, or solvate thereof and trabectedin, or a salt, hydrate, or solvate thereof, for use in a method of treatment, for example, of a disease or disorder which is mediated by histone deacetylase (HDAC) (for example, cancer, including, for example, ovarian cancer, soft-tissue sarcoma, osteosarcoma, etc.).

Another aspect of the present invention relates to use of belinostat, or a salt, hydrate, or solvate thereof, in the manufacture of a medicament, for use in combination with trabectedin, or a salt, hydrate, or solvate thereof in the treatment of, for example, a disease or disorder which is mediated by histone deacetylase (HDAC) (for example, cancer, including, for example, ovarian cancer, soft-tissue sarcoma, osteosarcoma, etc.).

Another aspect of the present invention relates to use of trabectedin, or a salt, hydrate, or solvate thereof, in the manufacture of a medicament, for use in combination with belinostat, or a salt, hydrate, or solvate thereof in the treatment of, for example, a disease or disorder which is mediated by histone deacetylase (HDAC) (for example, cancer, including, for example, ovarian cancer, soft-tissue sarcoma, osteosarcoma, etc.).

Another aspect of the present invention relates to use of a combination of belinostat, or a salt, hydrate, or solvate thereof and trabectedin, or a salt, hydrate, or solvate thereof, in the manufacture of a medicament, for use in the treatment of, for example, a disease or disorder which is mediated by histone deacetylase (HDAC) (for example, cancer, including, for example, ovarian cancer, soft-tissue sarcoma, osteosarcoma, etc.).

As will be appreciated by one of skill in the art, features and preferred embodiments of one aspect of the invention will also pertain to other aspects of the invention.

### BRIEF DESCRIPTION OF THE DRAWINGS

FIG. 1 is a bar-graph showing % survival of cells in a clonogenic assay following treatment with various concentrations of belinostat alone, trabectedin alone, and belinostat with trabectedin in the ovarian cancer cell line A2780.

FIG. 2 is a bar-graph showing % survival of cells in a clonogenic assay following treatment with various concentrations of belinostat alone, trabectedin alone, and belinostat with trabectedin in the sarcoma cell line MesSa.

FIG. 3 is a bar-graph showing % survival of cells in a clonogenic assay following treatment with various concentrations of belinostat alone, trabectedin alone, and belinostat with trabectedin in the osteosarcoma cell line Saos-2.

FIG. 4 is a graph of median tumour volume (mm³) as a function of time (days) for treatment with vehicle, belinostat alone, trabectedin alone, and belinostat with trabectedin, over the course of a 21-day xenograft (sarcoma cell line MesSa) study in nude mice.

FIG. 5 is a bar-graph showing median tumour weight (mg) at the end of a 21-day xenograft (sarcoma cell line MesSa) study in nude mice following treatment with vehicle, belinostat alone, trabectedin alone, and belinostat with trabectedin.

FIG. 6 is a graph of median body weight (g) as a function of time (days) for treatment with vehicle, belinostat alone, trabectedin alone, and belinostat with trabectedin, over the course of a 21-day xenograft (sarcoma cell line MesSa) study in nude mice.

### DETAILED DESCRIPTION OF THE INVENTION

The present invention relates to the surprising and unexpected discovery that the combination of (a) belinostat, or a salt, hydrate, or solvate thereof and (b) trabectedin, or a salt, hydrate, or solvate thereof, is synergistic, for example, in the treatment of diseases and disorders which are mediated by histone deacetylase (HDAC) (for example, cancer, including, for example, ovarian cancer, soft-tissue sarcoma, osteosarcoma, etc.).

Described herein is a method of treatment, for example, of a disease or disorder which is mediated by histone deacetylase (HDAC) (for example, cancer, including, for example, ovarian cancer, soft-tissue sarcoma, osteosarcoma, etc.) in a patient in need of treatment, comprising administering to said patient (a) belinostat, or a salt, hydrate, or solvate thereof, and (b) trabectedin, or a salt, hydrate, or solvate thereof, in amounts such that the combination is therapeutically-effective (e.g., in amounts such that the combination is therapeutically synergistic).

Also described herein is belinostat, or a salt, hydrate, or solvate thereof, for use, in combination with trabectedin, or a salt, hydrate, or solvate thereof, in a method of treatment of the human or animal body by therapy.

Also described herein is trabectedin, or a salt, hydrate, or solvate thereof, for use, in combination with belinostat, or a salt, hydrate, or solvate thereof, in a method of treatment of the human or animal body by therapy.

Also described herein is a combination of belinostat, or a salt, hydrate, or solvate thereof and trabectedin, or a salt, hydrate, or solvate thereof, for use in a method of treatment of the human or animal body by therapy.

Also described herein is belinostat, or a salt, hydrate, or solvate thereof, for use, in combination with trabectedin, or a salt, hydrate, or solvate thereof, in a method of treatment, for example, of a disease or disorder which is mediated by histone deacetylase (HDAC) (for example, cancer, including, for example, ovarian cancer, soft-tissue sarcoma, osteosarcoma, etc.).

Also described herein is trabectedin, or a salt, hydrate, or solvate thereof, for use, in combination with belinostat, or a salt, hydrate, or solvate thereof, in a method of treatment, for example, of a disease or disorder which is mediated by histone deacetylase (HDAC) (for example, cancer, including, for example, ovarian cancer, soft-tissue sarcoma, osteosarcoma, etc.).

Also described herein is a combination of belinostat, or a salt, hydrate, or solvate thereof and trabectedin, or a salt, hydrate, or solvate thereof, for use in a method of treatment, for example, of a disease or disorder which is mediated by histone deacetylase (HDAC) (for example, cancer, including, for example, ovarian cancer, soft-tissue sarcoma, osteosarcoma, etc.).

Also described herein is use of belinostat, or a salt, hydrate, or solvate thereof, in the manufacture of a medicament for use in combination with trabectedin, or a salt, hydrate, or solvate thereof in the treatment of, for example, a disease or disorder which is mediated by histone deacetylase (HDAC) (for example, cancer, including, for example, ovarian cancer, soft-tissue sarcoma, osteosarcoma, etc.).

Also described herein is use of trabectedin, or a salt, hydrate, or solvate thereof, in the manufacture of a medicament for use in combination with belinostat, or a salt, hydrate, or solvate thereof in the treatment of, for example, a disease or disorder which is mediated by histone deacetylase (HDAC) (for example, cancer, including, for example, ovarian cancer, soft-tissue sarcoma, osteosarcoma, etc.).

Also described herein is use of a combination of belinostat, or a salt, hydrate, or solvate thereof and trabectedin, or a salt, hydrate, or solvate thereof, in the manufacture of a medicament, for use in the treatment of, for example, a disease or disorder which is mediated by histone deacetylase (HDAC) (for example, cancer, including, for example, ovarian cancer, soft-tissue sarcoma, osteosarcoma, etc.).

Treatment Order and Timing

Typically, the treatment is performed over one or more treatment cycles, wherein the active agents, (a) belinostat, or a salt, hydrate, or solvate thereof, and (b) trabectedin, or a salt, hydrate, or solvate thereof, are administered to the patient over the course of each of said treatment cycles.

Within a treatment cycle, the active agents, (a) belinostat, or a salt, hydrate, or solvate thereof, and (b) trabectedin, or a salt, hydrate, or solvate thereof, may be administered simultaneously, or sequentially (and if sequentially, in any order).

The treatment may comprise one treatment cycle, or two or more treatment cycles, which may be the same or different. For example, if there are two treatment cycles, they may, independently, have the same or different duration, the same or different treatment order, the same or different dosages, etc.

The number of treatment cycles may be, for example, from 2 to 6 (e.g., 2, 3, 4, 5, 6); for example, from 2 to 3 cycles; from 2 to 4 cycles; from 2 to 5 cycles; from 2 to 6 cycles; from 3 to 4 cycles; from 3 to 5 cycles; from 3 to 6 cycles; from 4 to 5 cycles; from 4 to 6 cycles; etc.

Any or each treatment cycle may be, for example, from 3 to 49 days in length; for example, about 3 days in length; about 7 days in length; about 14 days in length; about 21 days in length, about 28 days in length; about 35 days in length, about 42 days in length, about 49 days in length, etc.

Dosage

It will be appreciated by one of skill in the art that appropriate dosages of the active agents (belinostat, or a salt, hydrate, or solvate thereof; and trabectedin, or a salt, hydrate, or solvate thereof), and compositions comprising the active agents, can vary from patient to patient.

Determining the optimal dosages will generally involve the balancing of the level of therapeutic benefit against any risk or deleterious side-effects. The selected dosage levels will depend on a variety of factors including, but not limited to, the activity of the agents, the route of administration, the time of administration, the rate of excretion of the agents, the duration of the treatment, other drugs, compounds, and/or materials used in combination, the severity of the condition, and the species, sex, age, weight, condition, general health, and prior medical history of the patient.

The amounts and routes of administration will ultimately be at the discretion of the physician, veterinarian, or clinician, although generally the dosages will be selected to achieve local concentrations at the site of action which achieve the desired effect without causing substantial harmful or deleterious side-effects.

Route of Administration

In one embodiment, one or both active agents (belinostat, or a salt, hydrate, or solvate thereof; and trabectedin, or a salt, hydrate, or solvate thereof) is administered parenterally. In one embodiment, both active agents are administered parenterally.

In one embodiment, one or both active agents (belinostat, or a salt, hydrate, or solvate thereof; and trabectedin, or a salt, hydrate, or solvate thereof) is administered intravenously. In one embodiment, both active agents are administered intravenously.

In one embodiment, one or both active agents (belinostat, or a salt, hydrate, or solvate thereof; and trabectedin, or a salt, hydrate, or solvate thereof) is administered by intravenous injection. In one embodiment, both active agents are administered by intravenous injection.

In one embodiment, one or both active agents (belinostat, or a salt, hydrate, or solvate thereof; and trabectedin, or a salt, hydrate, or solvate thereof) is administered by intravenous infusion. In one embodiment, both active agents are administered by intravenous infusion.

“Infusion” differs from “injection” in that the term “infusion” describes the passive introduction of a substance (e.g., a fluid, electrolyte, etc.) into a vein or tissues by gravitational force, whereas the term “injection” describes the active introduction of a substance into a vein or tissues by additional forces, e.g., the pressure in a syringe. Intravenous infusion is often referred to as “intravenous drip” or “i.v. drip”.

In one embodiment, one or both active agents (belinostat, or a salt, hydrate, or solvate thereof; and trabectedin, or a salt, hydrate, or solvate thereof) is administered orally. In one embodiment, both active agents are administered orally.

Salts and Solvates

It may be convenient or desirable to prepare, purify, and/or handle a corresponding salt of belinostat, for example, a pharmaceutically-acceptable salt.

Similarly, it may be convenient or desirable to prepare, purify, and/or handle a corresponding salt of trabectedin, for example, a pharmaceutically-acceptable salt.

Also, it may be convenient or desirable to prepare, purify, and/or handle a corresponding solvate of belinostat or solvate of a salt of belinostat.

Similarly, it may be convenient or desirable to prepare, purify, and/or handle a corresponding solvate of trabectedin or solvate of a salt of trabectedin.

Examples of pharmaceutically acceptable salts are discussed in Berge et al., 1977, “Pharmaceutically Acceptable Salts,” J. Pharm. Sci., Vol. 66, pp. 1-19.

Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄+).

Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

The term “solvate” is used herein in the conventional sense to refer to a complex of solute (e.g., belinostat, salt of belinostat, trabectedin, salt of trabectedin) and solvent. If the solvent is water, the solvate may be conveniently referred to as a hydrate, for example, a mono-hydrate, a di-hydrate, a tri-hydrate, etc.

Formulations

The belinostat and trabectedin may be provided, each separately or together in combination, in a formulation suitable for administration.

For example, while it is possible for each of belinostat and trabectedin to be administered alone, it is preferable to present them either together in combination, as a pharmaceutical formulation (e.g., composition, preparation, medicament) comprising both belinostat and trabectedin together with one or more other pharmaceutically acceptable ingredients well known to those skilled in the art, or as separate pharmaceutical formulations (e.g., compositions, preparations, medicaments) comprising belinostat or trabectedin, respectively, each together with one or more other pharmaceutically acceptable ingredients well known to those skilled in the art. Examples of pharmaceutically acceptable ingredients well known to those skilled in the art include, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation(s) may further comprise other active agents, for example, other therapeutic or prophylactic agents.

Thus, also described herein is a pharmaceutical composition comprising both belinostat and trabectedin, as defined above, and methods of making such a pharmaceutical composition comprising mixing belinostat and trabectedin together with one or more other pharmaceutically acceptable ingredients well known to those skilled in the art. If formulated as discrete units (e.g., tablets, etc.), each unit contains a predetermined amount (dosage) of belinostat and trabectedin.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., mammal, human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulation(s) may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation(s) may be prepared to provide for rapid or slow release; immediate, delayed, timed, or sustained release; or a combination thereof.

The formulation(s) may be prepared to provide a liposome or other microparticulate which is designed to target the belinostat and/or trabectedin, for example, to blood components or one or more organs.

The formulation(s) may suitably be in the form of a liquid, a solution (e.g., aqueous, non-aqueous), a suspension (e.g., aqueous, non-aqueous), an emulsions (e.g., oil-in-water, water-in-oil), etc.

The formulation(s) may suitably be in the form of suitable for parenteral administration (e.g., by injection, by infusion). Guidance for suitable parenteral formulations is provided, for example, in Avis et al., 1992. Formulations suitable for parenteral administration (e.g., by injection, by infusion), include aqueous or non-aqueous, isotonic, pyrogen-free, sterile liquids (e.g., solutions, suspensions), in which the belinostat and/or trabectedin is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Administration of Belinostat

In one embodiment, the belinostat, or a salt, hydrate, or solvate thereof, is administered parenterally.

In one embodiment, the belinostat, or a salt, hydrate, or solvate thereof, is administered intravenously.

In one embodiment, the belinostat, or a salt, hydrate, or solvate thereof, is administered by intravenous injection.

In one embodiment, the belinostat, or a salt, hydrate, or solvate thereof, is administered by intravenous infusion.

In one embodiment, the belinostat, or a salt, hydrate, or solvate thereof, is administered by prolonged intravenous infusion.

In one embodiment, the belinostat, or a salt, hydrate, or solvate thereof, is administered by prolonged continuous intravenous infusion.

By “prolonged”, it is intended that the intravenous infusion is for a period of at least about 12 hours.

By “continuous”, it is intended that the intravenous infusion is substantially uninterrupted, that is, continuous except for the requirements of administration, for example, the need to change reservoirs, i.v. bags, etc.

In one embodiment, the intravenous infusion (e.g., prolonged intravenous infusion, e.g., prolonged continuous intravenous infusion) is for a period of about 12 hours; of about 16 hours; of about 24 hours; of about 36 hours; of about 48 hours; of about 60 hours; or about 78 hours.

In one embodiment, the intravenous infusion (e.g., prolonged intravenous infusion, e.g., prolonged continuous intravenous infusion) is for a period of at least about 12 hours, for example, a period of from 12 to 24 hours, a period of from 12 to 48 hours, a period of from 12 to 60 hours, a period of from 12 to 72 hours, a period of from 12 to 96 hours, etc.

In one embodiment, the intravenous infusion (e.g., prolonged intravenous infusion, e.g., prolonged continuous intravenous infusion) is for a period of at least about 16 hours, for example, a period of from 16 to 24 hours, a period of from 16 to 48 hours, a period of from 16 to 64 hours, a period of from 16 to 72 hours, a period of from 16 to 96 hours, etc.

In one embodiment, the intravenous infusion (e.g., prolonged intravenous infusion, e.g., prolonged continuous intravenous infusion) is for a period of at least about 24 hours, for example, a period of from 24 to 48 hours, a period of from 24 to 72 hours, a period of from 24 to 96 hours etc.

In one embodiment, the intravenous infusion (e.g., prolonged intravenous infusion, e.g., prolonged continuous intravenous infusion) is for a period of at least about 36 hours, for example, a period of from 36 to 48 hours, a period of from 36 to 72 hours, a period of from 36 to 96 hours etc.

In one embodiment, the intravenous infusion (e.g., prolonged intravenous infusion, e.g., prolonged continuous intravenous infusion) is for a period of at least about 48 hours, for example, a period of from 48 to 72 hours, a period of from 48 to 96 hours etc.

In one embodiment, the intravenous infusion (e.g., prolonged intravenous infusion, e.g., prolonged continuous intravenous infusion) is for a period of at least 72 hours, for example, a period of from 72 to 96 hours etc.

Criteria for determining a suitable dosage of belinostat, or a salt, hydrate, or solvate thereof are discussed above (e.g., under the heading “Dosage”).

However, in general, a suitable dose of belinostat will be in the range of 100-2500 mg/m²/d, for example from 500-1500 mg/m²/d. Where the belinostat is provided as a salt, hydrate, or solvate, the amount administered is calculated on the basis of the parent compound and so the actual weight to be used is increased proportionately.

In one embodiment, for intravenous infusion (e.g., prolonged intravenous infusion, e.g., prolonged continuous intravenous infusion), the dosage during intravenous infusion is from 100 to 2500 mg/m²/d of belinostat.

In one embodiment, the lower end of the range is 300 mg/m²/d.

In one embodiment, the lower end of the range is 500 mg/m²/d.

In one embodiment, the lower end of the range is 700 mg/m²/d.

In one embodiment, the upper end of the range is 2000 mg/m²/d.

In one embodiment, the upper end of the range is 1500 mg/m²/d.

In one embodiment, the upper end of the range is 1300 mg/m²/d.

In one embodiment, the range is 300 to 2000 mg/m²/d.

In one embodiment, the range is 500 to 1500 mg/m²/d.

In one embodiment, the range is 700 to 1300 mg/m²/d.

Formulation of Belinostat

As discussed above, while it is possible for belinostat (or salt, hydrate, or solvate thereof) to be administered alone, it is preferable to present it as a pharmaceutical formulation (e.g., composition, preparation, medicament) comprising belinostat (or salt, hydrate, or solvate thereof) (and optionally also trabectedin) together with one or more other pharmaceutically acceptable ingredients well known to those skilled in the art.

In one embodiment, belinostat is employed.

In one embodiment, a salt of belinostat is employed.

In one embodiment, a hydrate of belinostat is employed.

In one embodiment, a hydrate of a salt of belinostat is employed.

As discussed above, the belinostat (or salt, hydrate, or solvate thereof) may be provided in a formulation suitable for parenteral administration, for example, a formulation suitable for administration by intravenous administration, e.g., intravenous infusion.

Belinostat, is sparingly soluble in water at physiological pH, and so must be administered in a pharmaceutical formulation where the belinostat is freely soluble and the composition is well tolerated, for example, in combination with L-arginine, as described in Bastin et al., 2006.

In one embodiment, the belinostat (or a salt, hydrate, or solvate thereof) is provided in a formulation suitable for parenteral administration (e.g., intravenously, e.g., by intravenous injection, intravenous infusion, etc.) further comprising L-arginine.

Typically, parenteral formulations (i.e., formulations suitable for parenteral administration, e.g., intravenous infusion) are typically packaged in plastic or glass large volume parenteral (LVP) containers to which is attached a suitable intravenous (i.v.) set at the time of infusion. Venous entry is typically by a metal needle or plastic catheter.

A continuous infusion system provides continuous regulated fluid flow at a pre-set rate. Once a prescribed flow rate (e.g., 125 mL/hr) has been established, the fluid should continue to flow accurately from the system until the reservoir container has emptied.

The infusion may be infused according to a continuous or intermittent dose schedule. A continuous schedule typically involves the non-stop infusion of a relatively large volume of fluid (e.g., 1 litre per 8 hour period for adults). Continuous therapy typically additionally provides fluid, electrolytes, agents to adjust acid-base balance, nutrients, and some other drugs. The total fluid intake must not exceed the patient's requirements (approximately 2400 mL per day for an adult).

Accordingly, the belinostat (or a salt, hydrate, or solvate thereof) may be formulated for parenteral administration, and may be presented, for example, in unit dose form in ampoules, pre-filled syringes, small volume infusion containers, or multi-dose containers optionally with an added preservative. The formulations may take such forms as suspensions, solutions or emulsions in oily or aqueous vehicles and may contain formulation agents such as suspending agents, stabilising agents, dispersing agents, etc.

For example, the belinostat may be provided as a concentrate for solution for infusion containing 50 mg/mL of belinostat and 100 mg/mL L-arginine, in water-for-injection, at a pH of 9.0-9.9. Immediately before administration, the concentrate is diluted, for example, with water-for-injection, glucose solution, or sodium chloride solution.

Alternatively, the belinostat may be formulated for oral administration, for example, at hard gelatin capsules (e.g., size 00) filled with belinostat (e.g., 250 mg).

Formulation and Administration of Trabectedin

As discussed above, while it is possible for trabectedin (or salt, hydrate, or solvate thereof) to be administered alone, it is preferable to present it as a pharmaceutical formulation (e.g., composition, preparation, medicament) comprising trabectedin (or salt, hydrate, or solvate thereof) (and optionally also belinostat) together with one or more other pharmaceutically acceptable ingredients well known to those skilled in the art.

In one embodiment, trabectedin is employed.

In one embodiment, a salt of trabectedin is employed.

In one embodiment, a hydrate of trabectedin is employed.

In one embodiment, a hydrate of a salt of trabectedin is employed.

As discussed above, the trabectedin may be provided in a formulation suitable for parenteral administration, for example, a formulation suitable for administration by intravenous administration, e.g., intravenous infusion.

Formulations of trabectedin (or a salt, hydrate, or solvate thereof) which are suitable for administration (e.g., administration intravenously, administration by intravenous infusion) are well-known in the art.

Criteria for determining a suitable dosage of trabectedin, or a salt, hydrate, or solvate thereof are discussed above (e.g., under the heading “Dosage”).

In addition, acceptable dosages of trabectedin (or a salt, hydrate, or solvate thereof) are well-known in the art, both in the context of treatments using trabectedin alone, and combination treatments using trabectedin with other active agents or therapies.

In one embodiment, the dosage of trabectedin corresponds to any of the well-known or standard dosages of trabectedin known in the art. For example, a known recommended dosage is 1.5 mg/m² administered as an intravenous infusion over 24 hours with a 3-week interval between cycles.

In one embodiment, for intravenous infusion, the dosage during intravenous infusion is from 0.1 to 5.0 mg/m²/d of trabectedin.

In one embodiment, the lower end of the range is 0.2 mg/m²/d.

In one embodiment, the lower end of the range is 0.5 mg/m²/d.

In one embodiment, the lower end of the range is 1 mg/m²/d.

In one embodiment, the upper end of the range is 4.0 mg/m²/d.

In one embodiment, the upper end of the range is 3.0 mg/m2/d.

In one embodiment, the upper end of the range is 2.0 mg/m²/d.

In one embodiment, the range is 0.2 to 4.0 mg/m²/d.

In one embodiment, the range is 0.5 to 3.0 mg/m²/d.

In one embodiment, the range is 1.0 to 2.0 mg/m²/d.

In one embodiment, the dosage is about 1.5 mg/m²/d.

In one embodiment, the dosage is about 1.1 mg/m²/d.

In one embodiment, the trabectedin is administered on day 1 of a 21 day cycle; e.g., as 1.5 mg/m²/d trabectedin by intravenous infusion on day 1 of a 21 day cycle; e.g., as 1.1 mg/m²/d trabectedin by intravenous infusion on day 1 of a 21 day cycle;

Conditions Treated

In one embodiment, the disease or disorder is a disease or disorder which is mediated by histone deacetylase (HDAC).

In one embodiment, the disease or disorder is a disease or disorder which is treatable or known to be treatable with an inhibitor of histone deacetylase (HDAC).

In one embodiment, the disease or disorder is a proliferative condition.

In one embodiment, the disease or disorder is a tumour.

In one embodiment, the disease or disorder is a solid tumour.

In one embodiment, the disease or disorder is cancer.

In one embodiment, the disease or disorder is solid tumour cancer.

In one embodiment, the disease or disorder is:
lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, neuroblastoma, glioma, sarcoma, osteosarcoma, bone cancer, nasopharyngeal cancer (e.g., head cancer, neck cancer), skin cancer, squamous cancer, Kaposi's sarcoma, melanoma, malignant melanoma, lymphoma, or leukemia.

In one embodiment, the disease or disorder is:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumour of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumour of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the disease or disorder is:
head cancer; neck cancer; nervous system cancer; brain cancer; neuroblastoma; lung/mediastinum cancer; breast cancer; oesophagus cancer; stomach cancer; liver cancer; biliary tract cancer; pancreatic cancer; small bowel cancer; large bowel cancer; colorectal cancer; gynaecological cancer; genito-urinary cancer; ovarian cancer; thyroid gland cancer; adrenal gland cancer; skin cancer; melanoma; bone sarcoma; soft-tissue sarcoma; paediatric malignancy; Hodgkin's disease; non-Hodgkin's lymphoma; myeloma; leukaemia; or metastasis from an unknown primary site.

In one embodiment, the disease or disorder is:
lung cancer, thymic cancer, thymoma, prostate cancer, renal cancer, liver cancer, bladder cancer, colorectal cancer, pancreatic cancer, gastric cancer, breast cancer, ovarian cancer, soft-tissue sarcoma, brain cancer, osteosarcoma, hepatocellular carcinoma, cancer of unknown primary (CUP), skin cancer, leukaemia, or lymphoma.

In one embodiment, the disease or disorder is soft-tissue sarcoma.

In one embodiment, the disease or disorder is: fibrosarcoma, myxofibrosarcoma, desmoid tumour, liposarcoma, synovial sarcoma, rhabdomyosarcoma, leiomyosarcoma, malignant peripheral nerve sheath tumours, angiosarcoma, gastrointestinal stromal tumour, Kaposi's sarcoma, Ewing's tumour, lyeolar soft part sarcoma, dermatofibromasarcoma protuberans, desmoplastic small round cell tumours, epithelioid sarcoma, extraskeletal myxoid chondrosarcoma, or giant cell fibrosarcoma.

In one embodiment, the disease or disorder is ovarian cancer.

In one embodiment, the disease or disorder is epithelial ovarian cancer.

In one embodiment, the disease or disorder is osteosarcoma.

The Patient

In one embodiment, the patient is a mammal, i.e., a living mammal. In one embodiment, the patient is a human, i.e., a living human, including a living human foetus, a living human child, and a living human adult.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included. For example, use with subjects who have not yet developed the condition, but who are at risk of developing the condition, is encompassed by the term “treatment.”

For example, treatment of a tumour may indicated by tumour reduction.

For leukaemia, “tumour reduction” may be indicated by a reduction in blast cells (e.g., the number of blast cells, the percentage of blast cells) in the blood (e.g., peripheral blood) and/or the reduction of blast cells (e.g., the number of blast cells, the percentage of blast cells) in the bone marrow.

For solid tumours, “tumour reduction” may be indicated by a reduction of tumour mass, for example, as determined by radiographic examination (e.g., using PET and/or NMR methods) or by physical examination.

The term “therapeutically-effective amount,” as used herein, pertains to the amounts of the active agents (belinostat, or a salt, hydrate, or solvate thereof; and trabectedin, or a salt, hydrate, or solvate thereof) that is effective for producing some desired therapeutic effect, commensurate with a reasonable benefit/risk ratio, when administered in accordance with a desired treatment regimen.

The term “therapeutically synergistic” as used herein, pertains to the amounts of the active agents (belinostat, or a salt, hydrate, or solvate thereof; and trabectedin, or a salt, hydrate, or solvate thereof) that are synergistic in respect of one or more desired therapeutic effects.

The term “treatment” includes combination treatments and therapies, in which two or more treatments or therapies are combined, for example, sequentially or simultaneously. For example, the active agents (belinostat, or a salt, hydrate, or solvate thereof; and trabectedin, or a salt, hydrate, or solvate thereof) may also be used in further combination therapies, e.g., in conjunction with other agents, for example, other cytotoxic agents, etc. Examples of further treatments and therapies include, but are not limited to, chemotherapy (the administration of other active agents, including, e.g., other HDAC inhibitors, antibodies (e.g., as in immunotherapy), prodrugs (e.g., as in photodynamic therapy, GDEPT, ADEPT, etc.); surgery; radiation therapy; and gene therapy.

Kits

One aspect of the invention pertains to a kit comprising (a) belinostat (or a salt, hydrate, or solvate thereof), or a composition comprising belinostat (or a salt, hydrate, or solvate thereof), e.g., preferably provided in a suitable container and/or with suitable packaging; and (b) instructions for use, e.g., written instructions on how to administer the compound or composition in accordance with the present invention, for example, in combination with trabectedin (or a salt, hydrate, or solvate thereof).

In one embodiment, the kit further comprises: (b) trabectedin, or a salt, hydrate, or solvate thereof, or a composition comprising trabectedin, or a salt, hydrate, or solvate thereof, e.g., preferably provided in a suitable container and/or with suitable packaging.

One aspect of the invention pertains to a kit comprising (a) trabectedin (or a salt, hydrate, or solvate thereof), or a composition comprising trabectedin (or a salt, hydrate, or solvate thereof), e.g., preferably provided in a suitable container and/or with suitable packaging; and (b) instructions for use, e.g., written instructions on how to administer the compound or composition in accordance with the present invention, for example, in combination with belinostat (or a salt, hydrate, or solvate thereof).

In one embodiment, the kit further comprises: (b) belinostat, or a salt, hydrate, or solvate thereof, or a composition comprising belinostat, or a salt, hydrate, or solvate thereof, e.g., preferably provided in a suitable container and/or with suitable packaging.

The written instructions may also include a list of indications for which the active ingredient(s) is/are a suitable treatment.

### EXAMPLES

The following examples are provided solely to illustrate the present invention and are not intended to limit the scope of the invention, as described herein.

### Study 1

### In Vitro Studies of Belinostat in Combination with Trabectedin

The effects of the combination of belinostat and trabectedin were studied in clonogenic assays. The clonogenic assay assesses anti-clonogenic activity of tumour cells. The “target” in clonogenic assay is all biochemical and molecular elements or alterations characteristic of tumour cells rather than specific single targets. Clonogenic assays are widely used in the development of anti-cancer agents and have been extensively used to characterize the anti-cancer effect of histone deacetylase (HDAC) inhibitors both as single agents (see, e.g., Kumagei et al., 2007; Hurtubise et al., 2006; Takai et al., 2004) and in combination with other anti-cancer therapies (see, e.g., Sarcar et al., 2010; Hurtubise et al., 2008; Chinnaiyan et al., 2008; Verheul et al., 2008; Geng et al., 2006; Boivin et al., 2002).

Method:

Cells were grown for at least 1 week after thawing in standard media to obtain a sub-confluent culture. The 3.3% agar was boiled for at least 60 minutes in a water bath on an electrical heating plate (30 mL PBS+990 mg Bacto agar). The 90 mL growth medium (RPMI-1640+10% FCS) was heated in a water bath at 37° C. Cells were centrifuged in 50 mL centrifuge tubes at 1200 rpm for 5 minutes at room temperature.

Drug (belinostat or trabectedin) was dissolved and diluted with growth medium or DMSO to a concentration of 300× the final concentration.

Cells were suspended in 7 mL growth medium using a 1 mL syringe and an 18 gauge needle by pumping the solution up and down 15 times. Cells were stained with Nigrosin (0.3 mL cells+0.3 mL 0.1% Nigrosin in PBS), and counted after 8 minutes using a Fuchs-Rosenthal counting chamber. Cells were diluted to obtain approximately 2000 colonies on untreated control plates, which, for most cell lines, gives 10,000 viable cells/m L.

Then, 10 mL agar and 90 mL growth medium was mixed (0.33%) and heated in a water bath at 37° C. 0.35 mL cell suspension was transferred to 10 mL conical centrifuge tubes using a dispenser. 35 μL drug (belinostat or trabectedin) was added. 3.15 mL agar/medium was added to each tube (maximum 8 tubes at the time).

For each belinostat combination tested series of 7 titrations corresponding to approximately 30, 40, 50, 60, 70, 80, 90% inhibition was tested as mono-therapy and in combination.

Ovarian Cancer Cells (A2780):

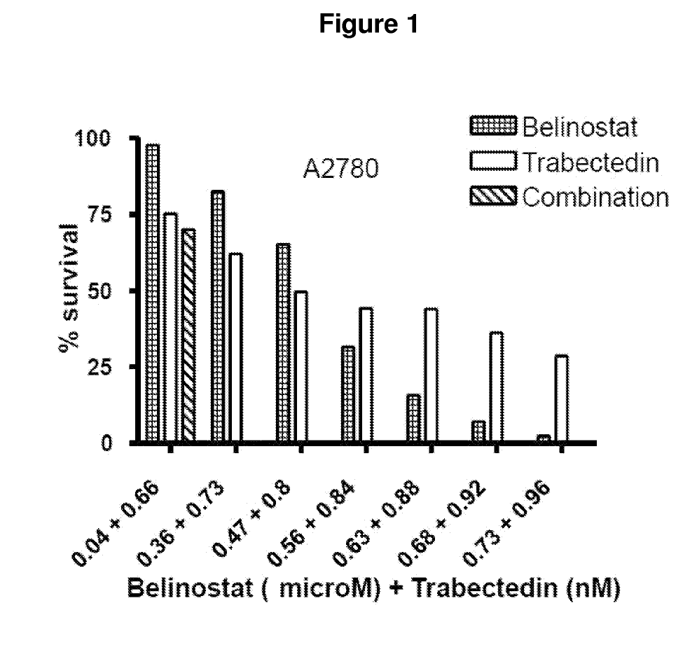

The results for the study using ovarian cancer cells (A2780) are summarised in the following table. The results are also illustrated in FIG. 1.

TABLE 1
Clonogenic Assay (Continuous Co-Incubation)
Ovarian Cancer Cells (A2780)

| | | Belinostat | Trabectedin | Belinostat with | |
|---|---|---|---|---|---|
| Belinostat | Trabectedin | Alone | Alone | Trabectedin | Combination |
| (μM) | (nM) | (% survival) | (% survival) | (% survival) | Index |
| 0.04 | 0.66 | 97.74 | 75.33 | 70.15 | 1.15 |
| 0.36 | 0.73 | 82.64 | 62.12 | 0.59 | 0.43 |
| 0.47 | 0.80 | 65.30 | 49.62 | 0.06 | 0.27 |
| 0.56 | 0.84 | 31.60 | 44.20 | 0.08 | 0.32 |
| 0.63 | 0.88 | 15.82 | 44.06 | 0.00 | 0.07 |
| 0.68 | 0.92 | 7.19 | 36.20 | 0.06 | 0.32 |
| 0.73 | 0.96 | 2.42 | 28.69 | 0.03 | 0.28 |

The Combination Index (CI) provides a measure of the interaction: A CI value of less than 1 indicates synergy, a CI value of 1 indicates an additive effect, and a CI value of greater than 1 indicates antagonism.

The data demonstrate that belinostat has a synergistic effect on ovarian cancer cells (A2780) in vitro in combination with trabectedin.

Sarcoma Cells (MesSa):

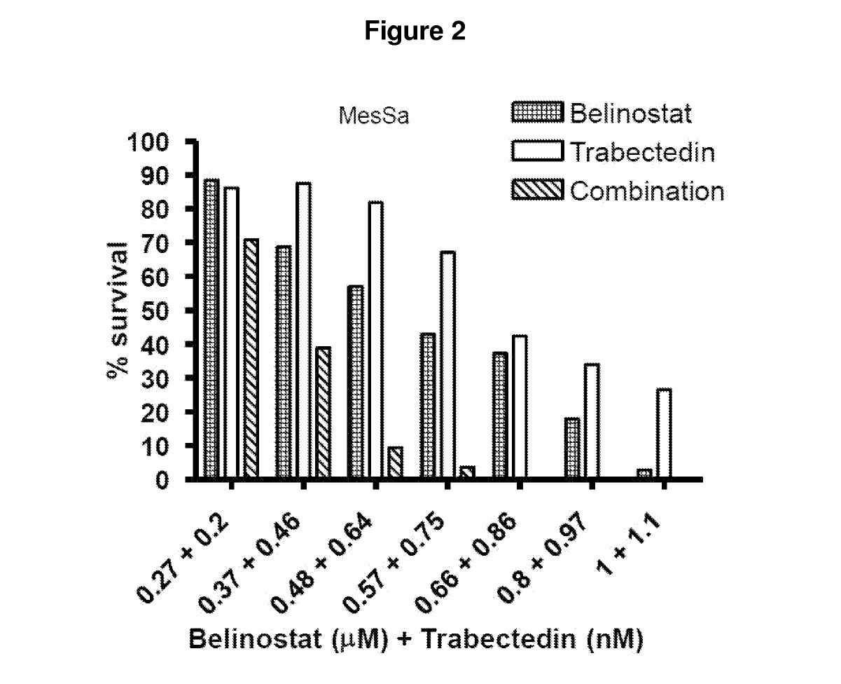

The results for the study using sarcoma cells (MesSa) are summarised in the following table. The results are also illustrated in FIG. 2.

TABLE 2
Clonogenic Assay (Continuous Co-Incubation)
Sarcoma Cells (MesSa)

| | | Belinostat | Trabectedin | Belinostat with | |
|---|---|---|---|---|---|
| Belinostat | Trabectedin | Alone | Alone | Trabectedin | Combination |
| (μM) | (nM) | (% survival) | (% survival) | (% survival) | Index |
| 0.27 | 0.20 | 88.48 | 86.17 | 70.83 | 1.07 |
| 0.37 | 0.46 | 68.82 | 87.51 | 38.91 | 1.07 |
| 0.48 | 0.64 | 57.06 | 81.84 | 9.36 | 0.73 |
| 0.57 | 0.75 | 42.92 | 67.10 | 3.65 | 0.62 |
| 0.66 | 0.86 | 37.33 | 42.38 | 0.19 | 0.29 |
| 0.80 | 0.97 | 17.90 | 33.98 | 0.00 | 0.04 |
| 1.0 | 1.1 | 2.75 | 26.55 | 0.02 | 0.23 |

The data demonstrate that belinostat has a synergistic effect on sarcoma cells (MesSa) in vitro in combination with trabectedin.

Osteocarcinoma Cells (Saos-2):

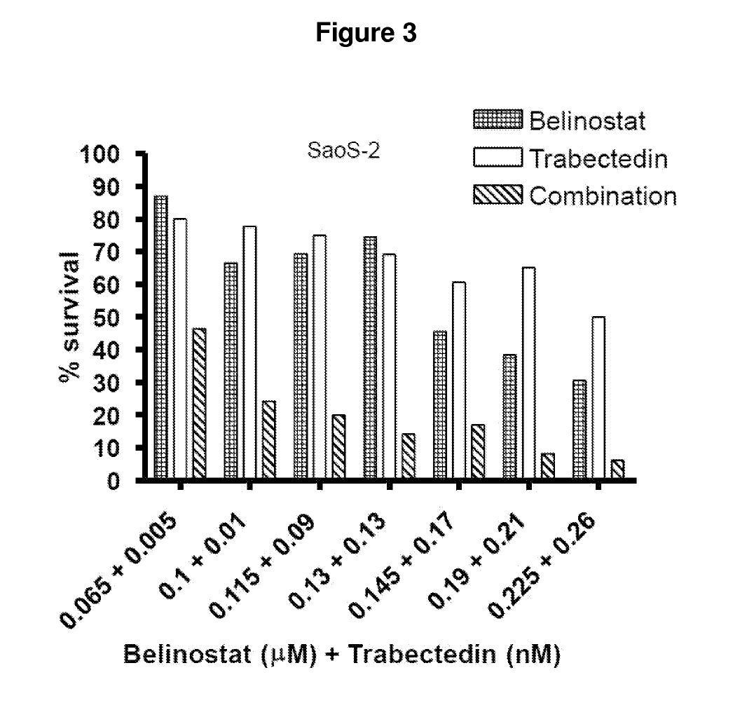

The results for the study using osteocarcinoma cells (Saos-2) are summarised in the following table. The results are also illustrated in FIG. 3.

TABLE 3
Clonogenic Assay (Continuous Co-Incubation)
Osteocarcinoma Cells (Saos-2)

| | | Belinostat | Trabectedin | Belinostat with | |
|---|---|---|---|---|---|
| Belinostat | Trabectedin | Alone | Alone | Trabectedin | Combination |
| (μM) | (nM) | (% survival) | (% survival) | (% survival) | Index |
| 0.065 | 0.005 | 86.96 | 79.99 | 46.36 | 0.39 |
| 0.10 | 0.010 | 66.47 | 77.74 | 24.22 | 0.38 |
| 0.115 | 0.09 | 69.29 | 74.94 | 20.02 | 0.39 |
| 0.13 | 0.13 | 74.52 | 69.14 | 14.15 | 0.36 |
| 0.145 | 0.17 | 45.50 | 60.61 | 17.05 | 0.45 |
| 0.19 | 0.21 | 38.34 | 65.08 | 8.17 | 0.40 |
| 0.225 | 0.26 | 30.59 | 49.99 | 6.09 | 0.41 |

The data demonstrate that belinostat has a synergistic effect on osteocarcinoma cells (Saos-2) in vitro in combination with trabectedin.

### Study 2

### In Vivo Studies of Belinostat in Combination with Trabectedin

The effects of belinostat in combination with trabectedin were also examined in a mouse xenograft model.

The xenograft model used the MesSa cell line (ATCC: CRL-1976 human sarcoma cells) (in vitro cell passage number 67 from frozen stock (number 65) on day 5). Tumour cells (1×10⁷) (trypsinated) (100 μL in 100 μL matrigel) were injected subcutaneously (s.c.) into the right flank of each mouse, with one node per mouse. The time from cell dilution to the end of injection was 24 minutes. Each mouse was anaesthetised with Hypnorm/Dormicum subcutaneously (s.c.) or isofluran (by inhalation) when the tumour cells were injected.

Four groups, each of 12 mice, were treated with vehicle, belinostat only, trabectedin only, or both belinostat and trabectedin, for three weeks, as summarised in the following table. On days that belinostat was administered, it was administered twice, with a 3 hour interval. On days that both belinostat and trabectedin were administered, the trabectedin was administered between the two belinostat administrations.

| Group | Treatment | Schedule |
|---|---|---|
| 1 | Vehicle: i.p. | twice, on days: 0-4, 7-11, 14-18 |
| | Vehicle: i.v. | once, on days: 0, 3, 7, 10, 14 |
| 2 | Belinostat: 40 mg/kg i.p. | twice, on days: 0-4, 7-11, 14-18 |
| 3 | Trabectedin: 0.1 mg/kg i.v. | once, on days: 0, 3, 7, 10 |
| 4 | Belinostat: 40 mg/kg i.p. | twice, on days: 0-4, 7-11, 14-18 |
| | Trabectedin: 0.1 mg/kg i.v. | once, on days: 0, 3, 7, 10 |

Mice were enrolled in treatment groups following confirmation of xenograft take. Both tumour diameter and body weight were measured on days: 0, 3, 7, 10, 14, 17, and 21. Animals were sacrificed (by cervical dislocation) on day 21. Tumour weight was measured on day 21 (following sacrifice).

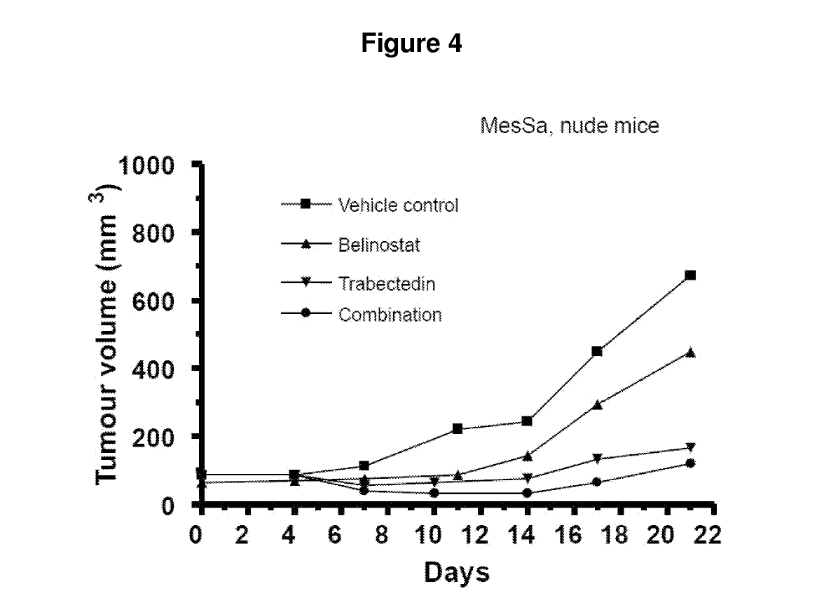

The tumour volume results are summarised in the following table. “T/C%” denotes tumour weight following treatment as a percent of control, using Mann-Whitney statistical analysis. The results (median tumour volume) are also illustrated in FIG. 4.

TABLE 4
Median Tumour Volume (mm³)

| | Vehicle | | Belinostat Only | | | Trabectedin Only | | | Belinostat & Trabectedin | | |
|---|---|---|---|---|---|---|---|---|---|---|---|
| Day | Vol. | N | Vol. | N | T/C % | Vol. | N | T/C % | Vol. | N | T/C % |
| 0 | 87 | 11 | 65 | 10 | 75 | 87 | 12 | 100 | 87 | 12 | 100 |
| 4 | 87 | 11 | 71 | 10 | 81 | 87 | 12 | 100 | 87 | 12 | 100 |
| 7 | 113 | 11 | 76 | 10 | 67 | 57 | 12 | 50 | 41 | 12 | 36 |
| 10 | 221 | 11 | 87 | 10 | 39 | 65 | 12 | 29 | 33 | 12 | 15 |
| 14 | 244 | 9 | 144 | 10 | 59 | 76 | 12 | 31 | 33 | 12 | 14 |
| 17 | 449 | 9 | 295 | 10 | 66 | 134 | 12 | 30 | 65 | 12 | 14 |
| 21 | 674 | 8 | 449 | 10 | 67 | 167 | 12 | 25 | 121 | 10 | 18 |
| p-val | | | | | 0.10 | | | 0.01 | | | 0.002 |

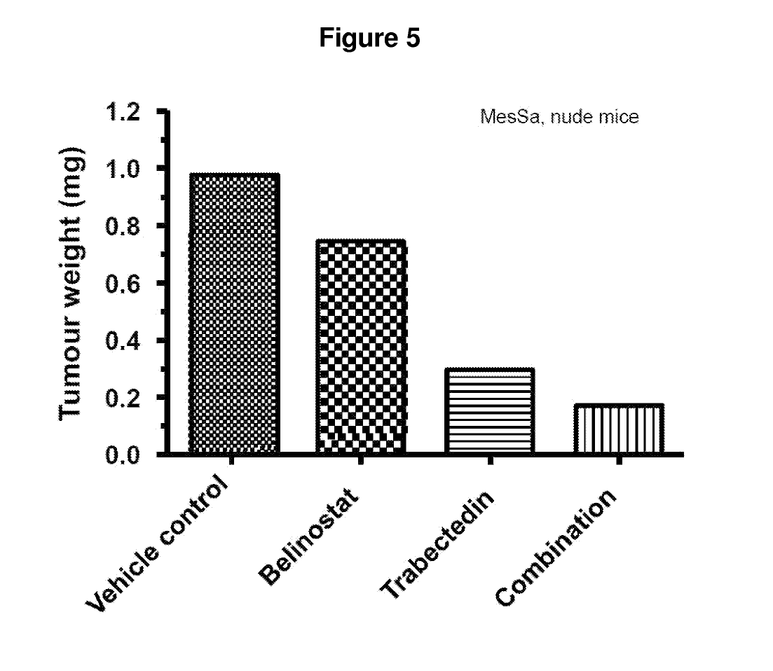

The tumour weight results are summarised in the following table. “T/C%” denotes tumour weight following treatment as a percent of control, using Mann-Whitney statistical analysis. The results (median tumour weight) are also illustrated in FIG. 5.

TABLE 5
Tumour Weight on Day 21

| | Tumour Weight (mg) | | | |
|---|---|---|---|---|
| | Vehicle | Belinostat | Trabectedin | Belinostat with |
| # | Only | Alone | Alone | Trabectedin |
| 1 | 1.253 | 0.018 | 0.763 | 0.138 |
| 2 | 0.893 | 0.711 | 0.333 | 0.203 |
| 3 | 1.950 | 0.522 | 0.153 | 0.051 |
| 4 | 0.201 | 0.323 | 0.588 | 0.075 |
| 5 | 0.470 | 0.794 | 0.075 | 0.430 |
| 6 | 0.042 | 1.052 | 0.726 | 0.185 |
| 7 | 1.436 | 0.250 | 0.058 | 0.172 |
| 8 | 1.053 | 1.060 | 0.030 | 1.202 |
| 9 | | 0.779 | 0.450 | 0.094 |
| 10 | | 0.972 | 0.257 | |
| Median | 0.973 | 0.745 | 0.295 | 0.172 |
| T/C % | | 77% | 30% | 18% |
| p-value | | 0.180 | 0.027 | 0.023 |

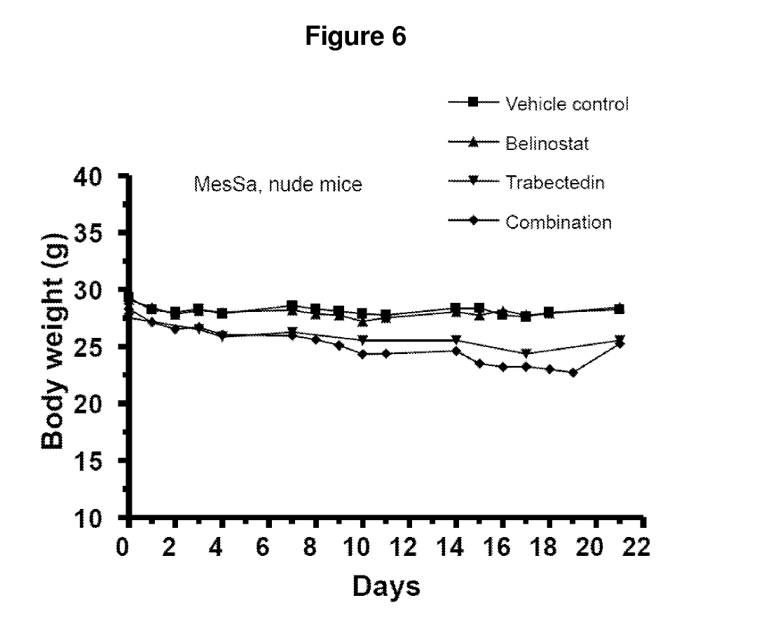

The median body weight results are illustrated in FIG. 6.

Belinostat (40 mg/kg, twice daily, i.p., 5 days per week for 3 weeks) caused some but not significant tumour growth delay in the MesSa human sarcoma xenograft model on nude mice. The T/C% for tumour volume on day 21 was 67% corresponding to 33% reduction in growth (p=0.10) (see Table 4). The T/C% for tumour weight on day 21 was 77% corresponding to 23% reduction in growth (p=0.180) (see Table 5). Belinostat was also well tolerated (see FIG. 6).

Trabectedin (0.1 mg/kg, once daily, i.v., on days 0, 3, 7, and 10 of a 3 week cycle) caused significant tumour growth delay in the MesSa human sarcoma xenograft model on nude mice. The T/C% for tumour volume on day 21 was 25% corresponding to 75% reduction in growth (p=0.01) (see Table 4). The T/C% for tumour weight on day 21 was 30% corresponding to 70% reduction in growth (p=0.027) (see Table 5). Trabectedin caused vein irritation and injections had to be stopped on day 10.

The combination of belinostat and trabectedin caused significant tumour growth delay in the MesSa human sarcoma xenograft model on nude mice. The T/C% for tumour volume on day 21 was 18% corresponding to 82% reduction in growth (p=0.002) (see Table 4). The T/C% for tumour weight on day 21 was 18% corresponding to 82% reduction in growth (p=0.023) (see Table 5). Some body weight loss (less than 20%) was observed in the group treated with the belinostat/trabectedin combination (see FIG. 6).

In summary, the data demonstrate a surprising and unexpected therapeutic synergy in soft-tissue sarcoma cells, osteosarcoma cells, and ovarian cancer cells, for the combination of belinostat with trabectedin.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below.

Each of these references is incorporated herein by reference in its entirety into the present disclosure, to the same extent as if each individual reference was specifically and individually indicated to be incorporated by reference.
Ali Elsayed et al., 2007, “Anticancer treatments”, international patent application publication number WO 2007/134203 A2 published 22 Nov. 2007.
Avis et al. (editors), 1992, “Pharmaceutical Dosage Forms: Parenteral Medications, Volume 1”, second edition, pp. 514-518.
Bastin et al., 2006, “Pharmaceutical formulations of HDAC inhibitors”, international patent application publication number WO 2006/120456 published 16 Nov. 2007.
Boivin et al., 2002, “Antineoplastic action of 5-aza-2′-deoxycytidine and phenylbutyrate on human lung carcinoma cells”, Anticancer Drugs, Vol. 13, No. 8, pp. 869-874.
Carter et al., 2010, “Trabectedin: A review of its use in soft tissue sarcoma and ovarian cancer”, Drugs, Vol. 70, No. 3, pp. 355-376.
Chinnaiyan et al., 2008, “Postradiation sensitization of the histone deacetylase inhibitor valproic acid”, Clin. Cancer Res., Vol. 14, No. 17, pp. 5410-5415.
D'Incalci et al., 2004, “Combination use of ecteinascidin-743 and platinum antineoplastic compounds”, international patent application publication number WO 2004/105761 Al published 9 Dec. 2004.
Geng et al., 2006, “Histone deacetylase (HDAC) inhibitor LBH589 increases duartion of gamma-H2AX foci and confines HDAC4 to the cytoplasm in irradiated non-small cell lung cancer”, Cancer Res., Vol. 66, No. 23, pp. 11298-11304.
Hurtubise et al., 2006, “Effect of histione deacetylase inhibitor LAQ824 on antineoplastic action of 5-Aza-2′-deoxycytidine (decitabine) on human breast carcinoma cells”, Cancer Chemother. Pharmacol., Vol 58, No. 5, pp. 618-625.
Hurtubise et al., 2008, “Preclinical evaluation of the antineoplastic action of 5-aza-2′-deoxycytidine and different histone deacetylase inhibitors on human Ewing's sarcoma cells”, Cancer Cell. Int., Vol. 8. p. 16.
Kumagai et al., 2007, “Histone deacetylase inhibitor, suberoylanilide hydroxamic acid (Vorinostat, SAHA) profoundly inhibits the growth of human pancreatic cancer cells”, Int. J. Cancer, Vol. 121, No. 3, pp. 656-665.
Lichenstein et al., 2012, “Combination therapies using HDAC inhibitors”, Australian patent application number AU 2012202000 Al published 3 May 2012.
Qian et al., 2006, “Activity of PXD101, a histone deacetylase inhibitor, in preclinical ovarian cancer studies”, Mol. Cancer Ther., Vol. 5, No. 8, pp. 2086-2095.
Rinehart et al., 1987, “Novel Compounds having antibacterial and antitumour properties” international patent application publication number WO 87/07610 published 17 Dec. 1987.
Ritchie et al., 2003, “The histone deacetylase inhibitor PXD101 synergises with established chemotherapeutics to inhibit tumor cell proliferation and upregulate apoptosis in vitro”, Clin. Cancer Research, Vol. 9, December 2003 (Supplement), Poster Session A, abstract number #A150, pages 6105s-6106s.
Rowinsky et al., 2005, “Combination therapy comprising the use of ET-743 and paclitaxel for treating cancer”, international patent application publication number WO 2005/049030 A1 published 2 Jun. 2005.
Ruffles et al., 2005, “Combination therapy comprising the use of ET-743 and doxorubicin for treating cancer”, international patent application publication number WO 2005/049029 A1 published 2 Jun. 2005.
Ruffles et al., 2005, “Combination”, international patent application publication number WO 2005/049031 A1 published 2 Jun. 2005.
Sarcar et al., 2010, “Vorinostat enhances the cytotoxic effects of the topoisomerase I inhibitor SN38 in glioblastoma cell lines”, J. Neurooncol., Vol. 99, No. 2, pp. 201-207.
Sehested et al., 2009, “Methods of treatment employing prolonged continuous infusion of belinostat”, international patent application publication number WO 2009/109861 published 11 Sep. 2009.
Takai et al., 2004, “Histone deacetylase inhibitors have a profound antigrowth activity in endometrial cancer cells”, Clin. Cancer Res., Vol. 10, No. 3, pp. 1141-1149.
Verheul et al., 2008, “Combination strategy targeting the hypoxia inducible factor-1 alpha with mammalian target of rapamycin and histone deacetylase inhibitors”, Clin. Cancer Res., Vol 14, No. 11, pp. 3589-3597.
Watkins et al., 2002, “Carbamic acid compounds comprising a sulfonamide linkage as HDAC inhibitors”, international patent application publication number WO 02/30879 A2 published 18 Apr. 2002.

1-66. (canceled)

67. A method of treatment of a disease or disorder in a patient in need of treatment, comprising administering to said patient:
(a) belinostat, or a salt, hydrate, or solvate thereof, and
(b) trabectedin, or a salt, hydrate, or solvate thereof, in amounts such that the combination is therapeutically effective;
wherein said disease or disorder is: soft-tissue sarcoma; fibrosarcoma; myxofibrosarcoma; desmoid tumour; liposarcoma; synovial sarcoma; rhabdomyosarcoma; leiomyosarcoma; malignant peripheral nerve sheath tumours; angiosarcoma; gastrointestinal stromal tumour; Kaposi's sarcoma; Ewing's tumour; lyeolar soft part sarcoma; dermatofibromasarcoma protuberans; desmoplastic small round cell tumours; epithelioid sarcoma; extraskeletal myxoid chondrosarcoma; giant cell fibrosarcoma; ovarian cancer; epithelial ovarian cancer; or osteosarcoma.

68. A method according to claim 67, wherein the disease or disorder is soft-tissue sarcoma.

69. A method according to claim 67, wherein the disease or disorder is fibrosarcoma, myxofibrosarcoma, desmoid tumour, liposarcoma, synovial sarcoma, rhabdomyosarcoma, leiomyosarcoma, malignant peripheral nerve sheath tumours, angiosarcoma, gastrointestinal stromal tumour, Kaposi's sarcoma, Ewing's tumour, lyeolar soft part sarcoma, dermatofibromasarcoma protuberans, desmoplastic small round cell tumours, epithelioid sarcoma, extraskeletal myxoid chondrosarcoma, or giant cell fibrosarcoma.

70. A method according to claim 67, wherein the disease or disorder is ovarian cancer.

71. A method according to claim 67, wherein the disease or disorder is epithelial ovarian cancer.

72. A method according to claim 67, wherein the disease or disorder is osteosarcoma.

73. A method according to claim 67, wherein said treatment comprises one or more treatment cycles, where said (a) belinostat, or a salt, hydrate, or solvate thereof, and (b) trabectedin, or a salt, hydrate, or solvate thereof, are administered to said patient over the course of each of said treatment cycles.

74. A method according to claim 73, wherein said treatment comprises from 2 to 6 treatment cycles.

75. A method according to claim 74, wherein said treatment cycle or each of said treatment cycles is from 3 to 49 days in length.

76. A method according to claim 74, wherein said treatment cycle or each of said treatment cycles is about 21 days in length.

77. A method according to claim 67, wherein said belinostat, or a salt, hydrate, or solvate thereof, is administered intravenously.

78. A method according to claim 67, wherein said belinostat, or a salt, hydrate, or solvate thereof, is administered by intravenous infusion.

79. A method according to claim 67, wherein said belinostat, or a salt, hydrate, or solvate thereof, is administered by prolonged continuous intravenous infusion.

80. A method according to claim 67, wherein said belinostat, or a salt, hydrate, or solvate thereof, is administered by continuous intravenous infusion for a period of from 12 to 72 hours.

81. A method according to claim 67, wherein said belinostat, or a salt, hydrate, or solvate thereof, is administered by continuous intravenous infusion for a period of about 48 hours.

82. A method according to claim 67, wherein the dosage of said belinostat, or a salt, hydrate, or solvate thereof, during said intravenous infusion is from 100 to 2500 mg/m²/d of belinostat.

83. A method according to claim 67, wherein the dosage of said belinostat, or a salt, hydrate, or solvate thereof, during said intravenous infusion is from 500 to 1500 mg/m²/d of belinostat.

84. A method according to claim 67, wherein said belinostat, or a salt, hydrate, or solvate thereof is administered orally.

85. A method according to claim 67, wherein said trabectedin, or a salt, hydrate, or solvate thereof, is administered intravenously.

86. A method according to claim 67, wherein the dosage of trabectedin, or a salt, hydrate, or solvate thereof, is 0.1 to 5 mg/m²/d administered intravenously.
